(19) United States
(12) Patent Application Publication
(10) Pub. No.: US 2013/0178776 A1
(43) Pub. Date: Jul. 11, 2013

(54) LATERAL ANKLE SUPPORT

(71) Applicant: Larry Fitzpatrick, Milton, MA (US)

(72) Inventor: Larry Fitzpatrick, Milton, MA (US)

(21) Appl. No.: 13/723,446

(22) Filed: Dec. 21, 2012

Related U.S. Application Data
(60) Provisional application No. 61/579,741, filed on Dec. 23, 2011.

(51) Int. Cl. A61F 5/01

(52) U.S. Cl. CPC A61F 5/0127
USPC 602/27

(57) ABSTRACT

A lateral ankle support for treating acute and chronic ankle sprains comprises a minimally stretchable but flexible adhesive bandage having a generally rectangular fibula pad portion, a first strap portion extending from an end of the pad portion and a second strap portion extending from a side of the pad portion, the two strap portions defining an acute included angle. The support is adapted to be adhered to an ankle so that the pad portion extends posteriorly over the fibularis longus and brevis muscles, the first strap portion overlies the caleaneofibular ligament and the second strap portion covers the anterior talofibular ligament.

### CROSS-REFERENCE TO RELATED APPLICATIONS

The present application claims the benefit of U.S. Provisional Patent Application Serial No. 61/579,741, filed Dec. 23, 2011.

### BACKGROUND OF THE INVENTION

This invention has to do with one of the most common lower extremity injuries in all running sports, the lateral ankle sprain. It relates more particularly to a lateral ankle support which allows injured athletes to return to play faster and safer while reducing the chance of further injury.

Ankle sprains are perhaps the single most common injury in the athletic population. Ankle sprains are generally caused by sudden ankle inversion or eversion, often in combination with plantar flexion or dorsi flexion. Sprains may be classified according to either location or mechanism of injury. A lateral ankle sprain occurs when the ligaments on the lateral side of the ankle are torn as a result of an ankle inversion. The lateral collateral ligament (LCL) complex of the ankle attaches a lateral malleolus to the talus and calcaneus. The lateral ligament consists of three distinctive parts: the anterior talofibular ligament (ATFL), the calcaneofibular ligament (CFL) and the posterior talofibular ligament (PTFL). The lateral ankle sprain is a common affliction among contact sports in those sports including multi-directional movements of the athlete.

Lateral sprains are probably the most frequent injury in sports including running and jumping. The severity of lateral ankle sprains is classified according to grades. A grade 1 sprain usually involves the ATFL; a grade 2, involves the ATFL and the CFL; and a grade 3 sprain involves the AFTL, CFL, PTFL and, frequently also, the anterior tibiafibula ligaments. Grade 1 inversion ankle sprain is the most common type of sprain because of the anatomical structure of the LCL complex. Grade 2 inversion ankle sprains have a high incident among sports participants and cause a great deal of disability with many days of lost time. Grade 3 inversion ankle sprains are relatively uncommon in sports; when they do happen, however, they are extremely disabling.

Treatment of lateral ankle sprain involves three phases. Phase 1: control hemorrhaging, swelling, pain and spasm. Phase 2: decrease swelling permit secondary io healing to occur, restore full muscle contraction without pain, restore 50% pain-free movement. Phase 3: restore symptom-free full range of motion, power, endurance, speed, and agility. During all three phases multiple treatments are used in order to return the athlete to play. During phase 3, it is common practice that some type of ankle support be worn initially.

Ankle taping and bracing proves to have a stabilizing effect on unstable ankles without interfering with motor performance. The athletic trainer can either tape the ankle with a variety of tapes in order to provide support or, he/she can place the athlete in an ankle brace designed to provide lateral stability to the LCL. Following a rigid rehabilitation program, it is the hope that the athlete will no longer need to have the ankle taped or braced for lateral stability. However, in some cases, athletes with chronic ankle instability may need to be taped continually or wear a brace throughout the season.

Ankle taping and bracing function positively when running straight ahead. However, since most sports are multidirectional, taping and bracing inhibit the athlete from pivoting and cutting during the final phase of rehabilitation. Taping, even when done properly, loses approximately 40% of its lateral stability within twenty minutes of vigorous activity. Ankle braces may maintain their stability better throughout vigorous activity, but they will often loosen up over time and need to be adjusted several times during an athletic event. Athletes commonly put braces on improperly and complain about feeling uncomfortable in their shoes. They also claim ankle braces make they feel slower. Thus, many athletes experience frustration when they can't sprint full speed without pain, and cannot change directions without irritating the LOC complex.

### SUMMARY OF THE INVENTION

This invention aims to provide a lateral ankle support to provide additional support to the LCL, allowing the athlete to return to play without risking further injury. The anatomically correct shape and dimensions of this support allow it to adhere to the lateral aspect of the ankle. Once the support has been properly secured in place, the athletic trainer may apply a traditional lateral ankle sprain taping over the support. Another object of this invention is to provide a support of this type having anatomical markings which will allow the support to be properly placed over the ATFL and CFL.

An additional object of this invention is to provide such a support having a fibula pad which extends posteriorly over the fibularis longus and brevis muscles. The additional tension provided by the pad will cause these muscles to contract and enhance ankle stability.

A further object of the invention is to provide an ankle support comprising two straps extending from the fibula pad and which cover the ATFL and CFL which will biomechanically reduce the stress load placed upon the LCL during ankle inversion.

Yet another object of the invention is to provide such an ankle support which is relatively inexpensive and can be applied easily in a timely fashion.

Other objects will, in part, be obvious and will, in part, appear hereinafter.

The invention accordingly comprises the several steps and the relation of one or more of such steps with respect to each of the others, and the article possessing the features, properties, and the relations of elements, which are exemplified in the following io detailed disclosure, and the scope of the invention will be indicated in the claims.

Briefly, my lateral ankle support has the form of a disposable, minimally stretchable and flexible adhesive bandage comprising a generally rectangular fibula pad and two strap portions extending, respectively, from an end and a side of the pad, those two strap portions defining an included acute angle, preferably about 60° . In use, the is support is adhered to an injured ankle so that the pad extends posteriorly over the fibularis longus and brevis muscles so as to tension those muscles and so that the two straps overlie the ATFL and CFL to reduce the stress level placed on the LCL during ankle inversion.

The disposable lateral ankle support may be contained in a package and with its adhesive coating being protected by non-adherent backing strips. The non-adherent strips may be removed from the back of the support and the support positioned directly on the skin. Anatomical landmark(s) may be provided on the support in order to help an athletic trainer properly place the support on the patient's ankle.

Once the ankle stabilizing support is in place, the fibular pad will cover approximately the bottom three inches of the fibula shaft. A landmark in the form of an opening may be provided to receive the lateral malleolus and help to properly position the support. The first strap will start at the inferior pole of the lateral malleolus and extend downward over the CFL and wrap under the calcaneus and end in mid heel. The second strap will start from the inferior pole of the lateral malleolus and extend along the lateral longitudinal arch to the base of the fifth metatarsal head. From this location, the second strap will turn in a crescent, sweeping back toward the first strap. Preferably, the second strap covers the entire ATFL as well as the talus, namicular, cuboid and head of the fifth metatarsal. Once in place, this lateral ankle support may be overlaid by a traditional ankle taping, which will help relieve the tension on the lateral ankle complex. The lateral ankle support may be removed in conjunction with the ankle tape.

### BRIEF DESCRIPTION OF THE DRAWINGS

For a fuller understanding of the nature and objects of the invention, is reference should be made to the following detailed description taken in connection with the accompanying drawings, in which;

FIG. 1 is a diagrammatic view of a lateral ankle support incorporating the invention positioned on a patient's right ankle;

FIG. 2 is a top plan view, on a larger scale, of the support, and

FIG. 3 is a side elevational view thereof.

### DETAILED DESCRIPTION OF AN ILLUSTRATIVE EMBODIMENT

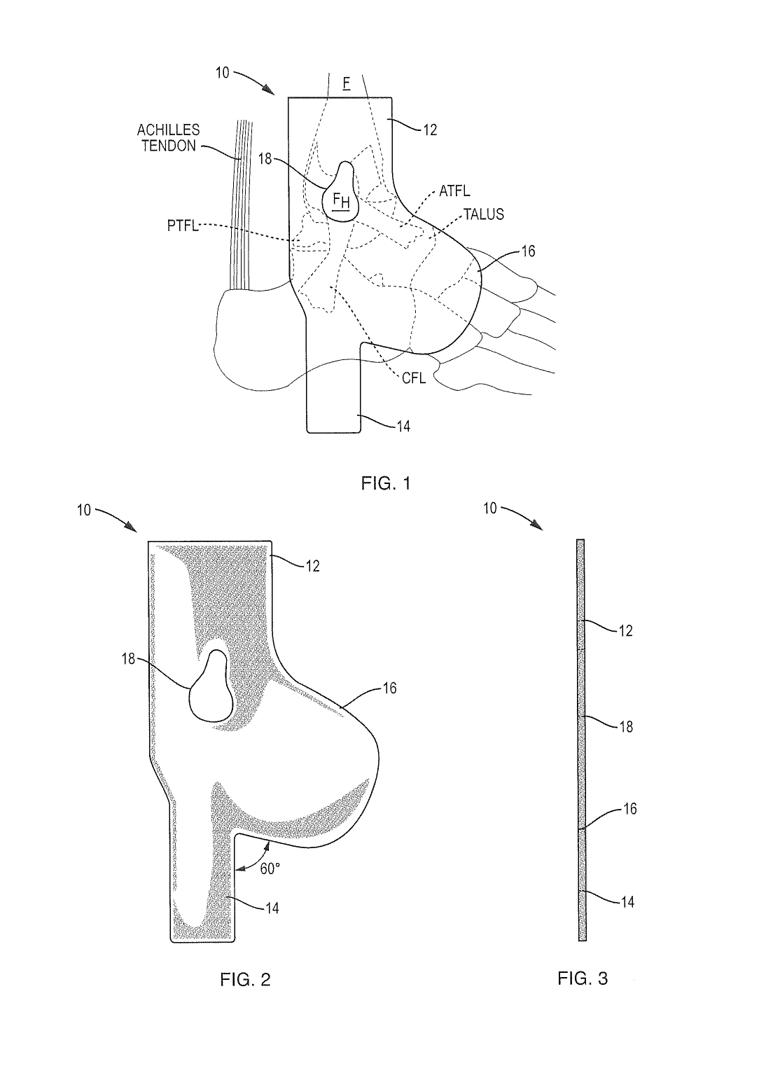

Refer now to FIGS. 1 to 3 of the drawings which show a lateral ankle support, indicated generally at 10 for a right ankle, the left ankle support being a mirror s image thereof. The support includes a fibula pad 12 which may be about three inches long and two inches wide with a thickness of about one-eighth inch and possess an adhesive coating on the back surface. The support 10 also includes a first strap 14 extending directly down from the lower end of pad 12 and adapted to cover the CFL. Typically, it is about three-quarter inch wide and three inches in length with a thickness io of one-eighth inch and also has an adhesive back surface. Finally, support 10 further includes a second strap 16 extending from a side of pad 12 and adapted to cover the ATLF of the underlying foot. Strap 16 is about three-quarter inch wide, three inches in length with a thickness of one-eighth inch; it also has an adhesive back surface.

When support 10 is properly applied to the ankle as shown in FIG. 1, the is pad 12 extends up on the fibula F about three inches from the distal lateral malleolus head F_{H} of the fibula F. An opening 18 may be provided in pad 12 to mark the location of the fibula head F_{H} when the support 10 is properly positioned on the ankle. When so placed, the strap 14 extends downward from the inferior pole of the lateral malleolus, with the portion thereof shown in phantom in FIG. 1 sweeping under the calcaneus and ending in the middle of the heel. The strap 16 starts from the inferior pole of the lateral malleolus and extends anteriorly along the lateral longitudinal and ends at the base of the fifth metatarsal head. From this location, strap 16 arcs upward and rearward, sweeping back toward the fibular strap at a 60° angle to the CFL. Thus, strap 16 covers the entire ATFL and the talus, namicular, cuboid and head of the fifth metatarsal.

The support may be made in multiple sizes, e.g., small, medium and large. Each support 10 may be individually packaged according to size, allowing easy access for the user.

Since the lateral ankle support 10 is constructed out of adhesive bandage-like material which will have minimal flexibility and will adhere to the skin, a traditional lateral ankle taping overwrap may help secure support 10 in place throughout the duration of the physical activity.

It is apparent from the foregoing that my lateral ankle support for treating acute and chronic ankle sprains will allow athletes to return to play faster while reducing the risk of further injury.

As discussed above, the lateral ankle support may be made of a material that will be malleable to the lateral ankle complex and move in conjunction with all ankle movements. This material should be porous and once in place have a minimal stretch factor in order to support the lateral ligamentous complex. Furthermore, the support material should be unaffected by water, or other moisture and maintain its function and form during vigorous activity.

The lateral ankle support described above is efficient and economical. Also, since certain changes may be made in the above construction without departing from the scope of the invention, it is intended that all matter contained in the above description or shown in the accompanying drawings shall be interpreted as illustrative and not in a limiting sense.

It should also be understood that the following claims are intended to cover all of the generic and specific features of the invention described herein.

What is claimed is:

1. A lateral ankle support comprising a minimally stretchable but flexible adhesive bandage having a generally rectangular fibula pad portion, a first strap portion extending from an end of the pad portion and a second strap portion extending from a side of the pad portion, said strap portions defining an acute included angle, said support adapted to be adhered to an ankle so that the pad portion extends posteriorly over the fibularis longus and brevis muscles, the first strap portion overlies the caleaneofibular ligament and the second strap portion covers the anterior talofibular ligament.

2. The support defined in claim 1 wherein the selected angle is 60°.

3. The support defined in claim 1 wherein the first strap portion is narrower than the pad portion and the second strap portion is wider than the pad portion.

4. The support defined in claim 1 and further including an ankle positioning landmark incorporated into the support.

5. The support defined in claim 4 wherein the landmark comprises an opening in the pad portion through which the ankle's fibula head is exposed when the support is positioned properly on the ankle.
